(19) United States
(12) Patent Application Publication
(10) Pub. No.: US 2025/0345380 A1
(43) Pub. Date: Nov. 13, 2025

(54) HERBAL EXTRACT COMPOSITION FOR THE TREATMENT OF OSTEOARTHRITIS AND CARTILAGE REGENERATION

(71) Applicant: Phytoveda Pvt. Ltd., Mumbai (IN)

(72) Inventors: Dilip Mehta, Mumbai (IN); Anselm de Souza, Mumbai (IN); Anirudh Dilip Mehta, Mumbai (IN); Ashit Harshad Vora, Mumbai (IN)

(21) Appl. No.: 19/201,106

(22) Filed: May 7, 2025

Related U.S. Application Data
(60) Provisional application No. 63/681,045, filed on Aug. 8, 2024.

(30) Foreign Application Priority Data
May 9, 2024 (IN) 202421036840

(51) Int. Cl. A61K 36/324; A61K 31/366; A61K 36/23; A61P 19/02

(52) U.S. Cl. CPC A61K 36/324; A61K 31/366; A61K 36/23; A61P 19/02; A61K 2236/15; A61K 2236/333; A61K 2236/35; A61K 2236/39

(57) ABSTRACT

Presently described are compositions comprising 55% w/w enriched Boswellia serrata extract containing 40% w/w total boswellic acids and 45% w/w enriched Apium graveolens L. seed extract containing 15% w/w celery bioactive (seselin and bergeptan) as quantified by HPLC, optionally along with pharmaceutically or nutraceutically acceptable excipients. The composition exhibits a synergistic effect while reducing cartilage degenerative biomarkers and increasing cartilage regenerative biomarkers when used for the treatment of osteoarthritis. The administration of the said composition also results in the reduction of pain, inflammation, and stiffness and an increase in the gap between knee joints of patients suffering from knee osteoarthritis. The methods of enrichment of extracts, preparation of said composition and treatment of subjects suffering from cartilage degeneration and osteoarthritis are described.

### CROSS-REFERENCE TO RELATED APPLICATIONS

This application is related to and claims priority from the Indian provisional application 20/242,1036840 filed May 9, 2024 and the U.S. provisional application 63/681,045 filed 8th August 2024, both of which are incorporated herein by reference in their entirety.

### BACKGROUND

1. Field. The present invention deals with a synergistic composition for the treatment of knee osteoarthritis.

2. Background Information. Osteoarthritis (OA) is a highly prevalent chronic joint disease. It is a progressively degenerating disease characterised by the loss of articular cartilage, hypertrophy of bones at the margins, subchondral sclerosis, and biochemical and morphological changes of the synovium. The most frequently diagnosed type of arthritis is knee osteoarthritis. Its incidence is continuously growing along with increasing life expectancy and obesity [1, 2]. Globally, knee OA accounts for nearly 80% of the total OA burden. Knee O A, also known as degenerative joint disease, results from progressive wear and tear of the joints. Research has revealed, ˜10% of men and 13% of women aged 60 years or more have characteristic knee OA. Not all trial participants radiographically exhibiting knee OA are symptomatic. The overall prevalence of knee OA was found to be 28.7% in India. The occurrence of symptomatic knee OA is ˜240 cases in 100,000 patients annually, regardless of age [3, 4].

Knee osteoarthritis is classified depending on its cause as either primary or secondary. Primary knee osteoarthritis is the result of articular cartilage degeneration (wear and tear) without any known reason. Secondary knee osteoarthritis is the consequence of articular cartilage degeneration due to known reasons such as posttraumatic, postsurgical, scoliosis, rickets, hemochromatosis, gout, pseudogout, acromegaly, rheumatoid arthritis, infectious arthritis, psoriatic arthritis, haemophilia, Paget disease, etc. The modifiable risk factors for knee OA include trauma (articular), occupational exposure, muscle weakness, poor diet, weight, and metabolic syndrome; the non-modifiable risk factors are gender, age, race, and genetics [1,5].

Although knee osteoarthritis is closely correlated with ageing, it is not simply a consequence of ageing but rather it is a disease. The synovium of the OA produces synovial fluid, which lubricates and nourishes the vascular cartilage of the knee. Articular cartilage is composed primarily of type II collagen, proteoglycans, chondrocytes, and synovial fluid. Healthy articular cartilage maintains an equilibrium between each component so that any degradation of cartilage is matched by synthesis. In OA, degradative enzymes or matrix metalloproteases (MMPs) are overexpressed, disrupting the equilibrium and resulting in collagen and proteoglycans being lost. In the early stages of OA, chondrocytes secrete tissue inhibitors of MMPs (TIMPs) and attempt to increase the synthesis of proteoglycans to match the degradative process. However, this reparative process is not sufficient and loss in equilibrium results in reduced proteoglycans despite its increased synthesis, synovial fluid content, the disorganized pattern of collagen, and ultimately leading to loss of articular cartilage elasticity. Macroscopically these changes result in cracking and fissuring of the cartilage and thus erosion of the articular surface. As a result of cartilage injury, the collagen matrix is microscopically degraded, which promotes chondrocyte proliferation and clustering. This leads to cartilage outgrowth ossification and the development of osteophytes, resulting in chondrocyte apoptosis. The clinical symptoms associated with OA are pain, with or without activity and weight-bearing; stiffness after inactivity leading to reduced range of motion of the joints [1, 6].

Evidence-based approaches to the treatment of knee OA include nonpharmacologic, pharmacologic, and surgical modalities targeted at relieving pain, improving joint function, and modifying risk factors for disease progression. Strengthening, self-management programs, low-impact aerobic exercises, and neuromuscular education, weight loss for trial participants with a BMI >25 are recommended for knee OA treatments [7]. First-line drug treatments for symptomatic knee OA are topical and oral analgesics and non-steroidal anti-inflammatory drugs (NSAIDS). Other common pharmacological interventions include intra-articular corticosteroids, hyaluronic acid, glutathione, chondroitin, vitamin, mineral, and collagen supplementation etc [6, 8].

The treatment of osteoarthritis has largely been restricted to drugs that alleviate the symptoms. Additionally, long-term use is associated with adverse events that might result in drastic outcomes such as gastrointestinal problems, cardiovascular problems, and adverse effects on the cartilage. However, this approach is not sufficient to eradicate the disease. Currently, there are not many proven therapeutics available in the market that have reparative effects on articular cartilage or foster new tissue formation, as well as effective bone healing. Newer therapeutics and approaches, such as regenerative medicine and biomaterials-based therapies, can provide a better solution by restoring the normalcy of joint structure, functions, and biomechanics. Although these remedies have been taken up into clinical trials for their potential use in OA therapy, success is still a way off [9, 10].

Phytoconstituent supplements have gained interest among clinicians and researchers as they demonstrate a continuing approach for management and inhibiting OA as combination treatments. A systematic review and meta-analysis suggested that this supplement provides clinically meaningful effects on pain and function in trial participants with hand, hip, or knee osteoarthritis. There has been evidence that dietary bioactive combinations can reduce inflammatory markers in OA patients and improve clinical symptoms [10, 11].

U.S. Pat. No. 5,494,668 discloses a method of treating degenerative musculoskeletal diseases such as rheumatoid arthritis and osteoarthritis in an animal, typically a human, involving administering to the animal, typically enterally, in a convenient dosage form, a therapeutically effective amount of the beneficiated extracts of the plants Ashwagandha, Sallai Guggul, Turmeric, and Ginger, in a predetermined proportion relative to each other with or without other biologically active inorganic ingredients. U.S. Pat. No. 6,224,871 discloses a dietary supplement for nutritionally promoting healthy joint function in human subjects. The supplement includes as a major ingredient a protein derived from enzymatic hydrolysis of collagen in combination with lesser proportions of glucosamine sulfate, gingko biloba, borage oil powder, turmeric, Boswelia serrata, ashwagandha, Piper nigrum extract and a herbal blend.

U.S. Pat. No. 6,541,045 discloses a herbal composition for combating inflammation, comprising therapeutically effective amounts of Japanese knotweed, Devil's claw, grapeskin, and syzygium. Also provided is an herbal composition for soothing muscles and joints, comprising therapeutically effective amounts of Japanese knotweed, N-acetyl D-glucosamine, chondroitin sulfate, D-glucosamine hydrochloride, methylsulfonylmethane, grapeskin, syzygium, and Devil's claws.

However, there still remains a need for compositions that would treat more effectively knee osteo arthritis with proven efficacy.

### BRIEF DESCRIPTION OF THE DRAWINGS

FIG. 1. HPTLC fingerprints of enriched Boswellia serrata extract and raw material.

FIG. 2. HPLC chromatograms of enriched Boswellia serrata extract.

FIG. 3. Bioactive enriched chemical constituents of Boswellia serrara extract 5 (Serratol, total Boswellic acids including, α-Boswellic acid, β-Boswellic acid, 3-O-Acetyl-α-boswellic acid, 3-O-Acetyl-β-boswellic acid, 11-Keto-β-boswellic acid, 3-O-Acetyl-11-keto-β-boswellic acid, and total Tirucallic acids including 3-Keto-tirucallic acid, 3-O-Acetyl-α-tirucallic acid, and 3-O-Acetyl-β-tirucallic acid).

FIG. 4. HPLC chromatograms of Apium graveolens L. extract.

FIG. 5. Bioactive enriched chemical constituents of Apium graveolens L. extract (Bergapten and Seselin).

FIGS. 6A, 6B, and 6C. Radiological X-ray images of the knee before and after PVD 06 treatment for participants a) #0104, b) #0224, c) #0103.

### DETAILED DESCRIPTION

With the preceding background, the present invention provides a herbal extract composition useful for cartilage regeneration and treatment of osteoarthritis, comprising enriched Boswellia serrata extract and enriched Apium graveolens L. seed extract. Its efficacy was established by conducting a randomised double-blind placebo-controlled trial, by recruiting patients suffering from mild to moderate knee osteoarthritis conditions.

Thus the present invention provides a herbal extract composition comprising a) enriched Boswellia serrata extract wherein the total Boswellic acids content is not less than 40% w/w, total tirucallic acids is not less than 15% w/w and Serratol is not less than 15% w/w; b) enriched Apium graveolens L. extract wherein bergapten and seselin are in the ratio of the Boswellia serrata extract to the Apium graveolens L. extract at a range of 55: 45% w/w and wherein the said composition exhibits a synergistic effect when used in the treatment of knee osteoarthritis.

In the clinical trial, it was surprisingly found that the said herbal extract composition exhibited a synergistic effect while suppressing the expression of inflammatory markers such as IL-6, hs-CRP, TNF-Alpha, ESR, IL-7, IL-1 and enhancing the expression of cartilage regenerative biomarkers such as PIIANP and PIICP.

The present invention is directed to a herbal extract composition enriched in Boswellic acid content wherein the total Boswellic acid content is not less than 40% w/w. Additionally, the present invention provides an enriched Apium graveolens L seed extract wherein the total Bergapten and Seselin content is in the range not less than 15%.

In one aspect, the present invention is directed to a herbal extract composition comprising a 1) an enriched Boswellia serrata extract containing not less than 40% by weight of total Boswellic acids and 2) enriched Apium graveolens L. seed extract nor less than 15% by weight of Bergapten and Seselin, wherein the ratio of enriched Boswellia serrata extract to enriched Apium graveolens L. seed extract is in the range of 55: 45% w/w that exhibits a synergistic effect in lowering inflammatory markers in knee osteoarthritis patients, particularly IL-6, hs-CRP, TNF-alpha, CTX-2, IL-1, ESR and IL-7 in the overall range of 28-70%. More particularly, the reduction of IL-6 was in the range of 63-70%, hs-CRP in the range of 42-60%, TNF-alpha in the range of 25-33%, CTX-2 in the range of 38-45%, ESR in the range of 40-50%, IL-7 in the range of 42-52%, IL-1 in the range of 28-35%.

The herbal extract composition of the invention when administered to a patient suffering from knee osteoarthritis, synergistically reduces the level of cartilage degeneration biomarkers in the range 29% to 50%. More particularly, the present invention is directed to a composition comprising 1) an enriched Boswellia serrata extract containing not less than 40% by weight of total Boswellic acids and 2) an enriched Apium graveolens L. extract 15% by weight of Bergapten and Seselin, that exhibits a synergistic effect in reducing cartilage degenerative biomarkers in the range of 30-55%. More particularly, the reduction of CTX-II in the range of 35-45%, more particularly in the range of 40-43%; cartilage oligomeric matrix protein (COMP) in the range of 30-40%, more particularly in the range of 37-40%; Matrix metalloproteinase-3 (MMP-3), procollagen II C-terminal propeptide (PIICP) and Serum N-propeptide of collagen IIA (PIIANP) in the range of 40-50%, more particularly in the range of 43-47%.

According to an embodiment, the present disclosure is directed to a composition comprising 1) an enriched Boswellia serrata extract containing not less than 40% by weight of total Boswellic acids and 2) an enriched Apium graveolens L. seed extract not less than 15% by weight of Bergapten and Seselin, which on treatment to a person in the need results in a notable increase in the gap between knee joints, which correlates with clinical improvement.

The present invention is also directed to a process for the extraction of a bioactive-enriched fraction of Boswellia serrata extract from Oleo gum resins of Boswellia serrata, which results in an enhancement of Boswellic acid content of the extract from 6% w/w to not less than 40% w/w. More particularly, the boswellic acid content extract is from 7% to 30% w/w.

The present invention is directed to a process for the extraction of a bioactive enriched fraction of Apium graveolens L. extract from the seeds of Apium graveolens L., which results in an enhancement of Bergapten and Seselin content of the extract from 2% to 18% w/w, more particularly 3% w/w to 15% w/w.

In another aspect, the present disclosure is directed to a composition comprising a blend of Boswellia serrata and Apium graveolens L. seed extract, along with optionally, pharmaceutically or nutraceutically inactive excipient in the form of a capsule and/or tablet.

In yet another aspect, the present disclosure is directed to a composition comprising a blend of Boswellia serrata and Apium graveolens L. seed extract which is administered to a patient suffering from knee osteoarthritis at a dose of 550 mg, consisting of 300 mg of B. serrata extract and 250 mg enriched Apium graveolens L. extract.

The invention is now illustrated with examples which, however, should not be construed to limit the scope of the present invention.

### Example 1

Enrichment of Boswellia serrata Extract

The process for isolation of enriched extract of B. serrata oleo gum resin comprises: (a) procurement of Boswellia serrata oleo gum resin, (b) successive grinding of the oleo gum resin to prepare coarse powder, (c) Allowing the coarse oleo gum resin to macerate or percolate in the presence of ethyl alcohol (hereafter referred to as alcohol, (resin:alcohol::1:4 w/w)) at room temperature for 24 hours. (d) extracting the resin with alcohol (resin:alcohol::1:4 w/w) at ambient temperature for 0.5 to 10 hours. (e) Repeating step (d) 5-6 times to obtain an alcoholic extract containing not less than 40% Boswellic acids (f) The residue obtained after Boswellic acid extraction is used to enrich fraction consisting of Serratol by further extracting it with Hexane on a silica gel column (g) distilling the solvent layer obtained in step (f) on a rotary evaporator to obtain an extract containing not less than 40% w/w total boswellic acids i.e. (1) β-Boswellia Acid, (2) α-Boswellic acid, (3) 3-O-Acetyl-β-boswellic acid, (4) 3-O-Acetyl-α-boswellic acid, (5) 11-Keto-β-boswellic acid, (6) 3-O-Acetyl-11-Keto-β-boswellic acid), 15% w/w tirucallic acids (3-Oxo-tirucallic acid, 3-O-acetyl-α-tirucallic acid, 3-O-acetyl-β-tirucallic acid) and 15% serrarol quantified by HPLC-PDA.

### Physicochemical Properties

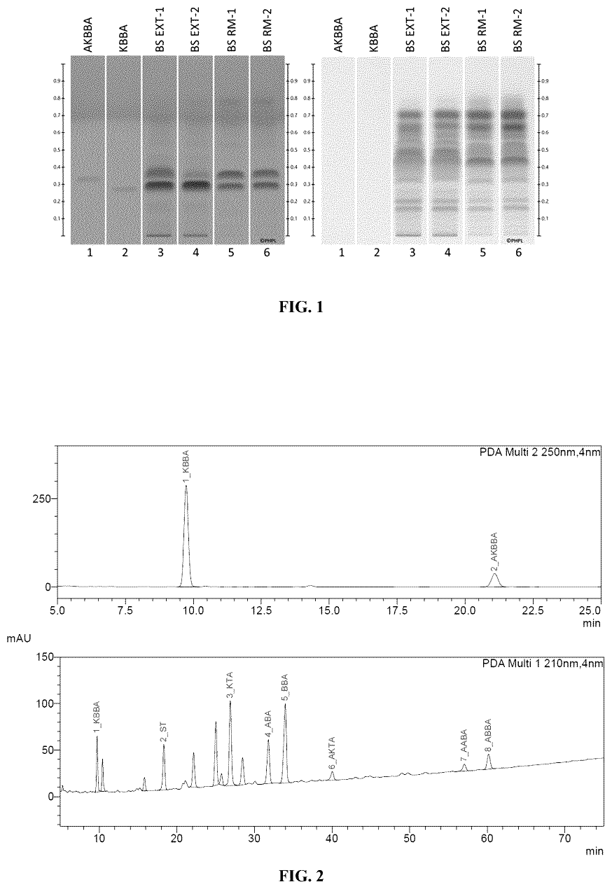

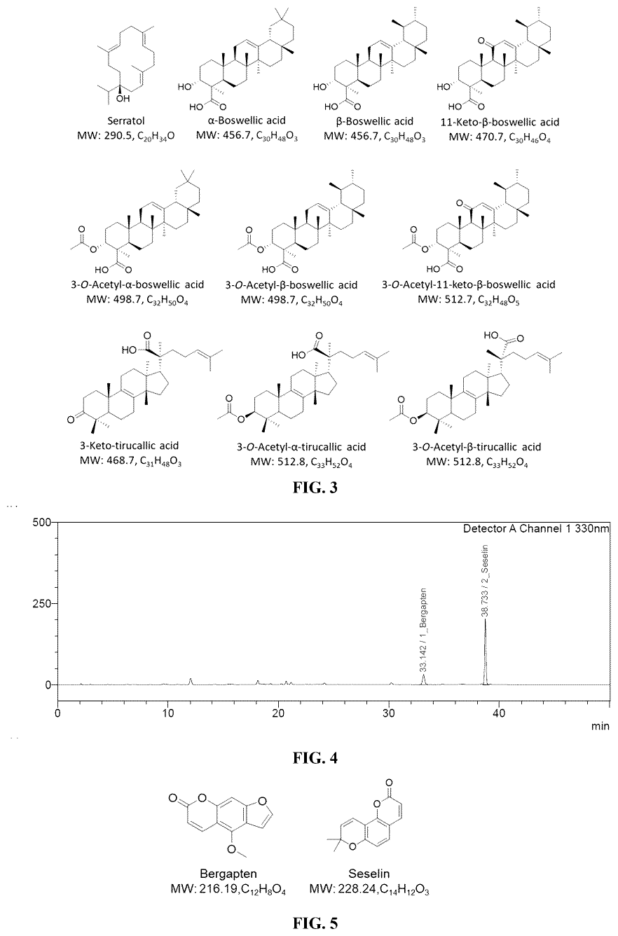

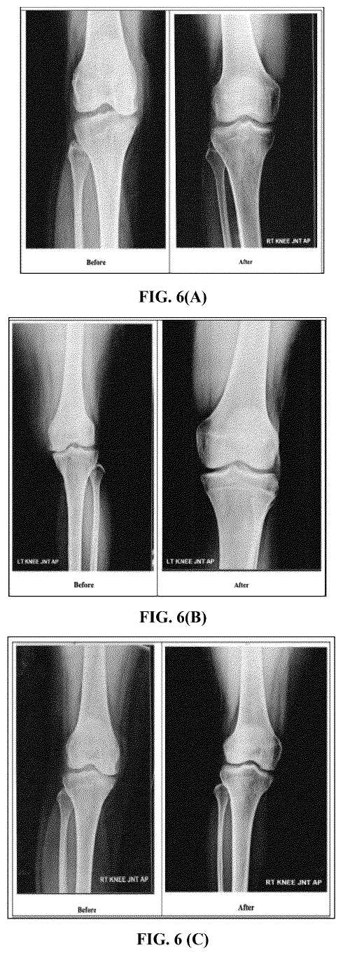

Boswellia serrata extract appears as a creamy-white powder with a characteristic odour. HPTLC fingerprints of Boswellia serrata extracts and raw materials demonstrate dark bands at Rf 0.28 and 0.34, representing KBBA (11-Keto-β-boswellic acid) and AKBBA (3-O-Acetyl-11-Keto-β-boswellic acid), respectively, under UV 254 nm as shown in FIG. 1. The HPLC chromatogram with characteristic major chemical components, serratol, total Boswellic acids, and total Tirucallic acids is shown in FIG. 2. The chemical structure of the bioactive extract of Boswellia serrata is shown in FIG. 3.

### Example 2

Enrichment of Apium graveolens L. Seed Extract

The process for enrichment of Apium graveolens L. seed extract comprises: (a) procurement of Apium graveolens L. seed, (b) successive grinding of the seeds to prepare coarse powder, and (c) allowing the coarse powder to macerate or percolate in the presence of the solvent at room temperature for 24 hours. (d) extracting the seeds with alcohol (seeds:alcohol: 1:4) at an ambient temperature for 0.5 to 10 hours. (e) repeating step d 5-6 times to obtain an alcoholic extract (f), concentrating the extract to isolate Apium graveolens L. extract comprising 0.60% to 5% w/w Bergapten and 3.15% to 20% w/w seselin as quantified by HPLC-PDA.

Apium graveolens extract appears as a brown colour powder with a characteristic odor. The HPLCchromatogram with characteristic major chemical components Bergapten, and Seselin, are shown in FIG. 4. Bioactive enriched chemical constituents of Apium graveolens L. extract (Bergapten and Seselin) are shown in FIG. 5.

### Example 3

Preparation of a Composition Comprising Boswellia Serrata Extract and Apium graveolens L. Seed Extract

Preparation of a composition comprising Boswellia serrata extract and Apium graveolens L. seed extract in the ratio 55% w/w to 45%. The steps involved: (a) Magnet pass and shifting of Boswellia serrata and Apium graveolens L. seed extract (b) compaction of the material (c) milling (d) shifitng (e) blending (f) passing through 20 mesh sieves (g) preparation of granules (f) metal magnet detection (g) packaging, labeling and storage of Material. The composition is cream to brown free-flowing granular powder with characteristic taste and odour, having a bulk density not less than 0.40 g/ml and a Tap Density not less than 0.50 g/ml.

### Example 4

Evaluation of Composition Comprising Boswellia Serrata Extract and Apium graveolens L. Seed Extract

Evaluation of composition comprising Boswellia serrata and Apium graveolens L. was carried out by conducting a randomised, double-blind, parallel-group, comparative, multicenter, placebo-controlled clinical trial to assess the efficacy and safety of Boswellia serrata and Apium graveolens (Celery) seed extract in the management of knee osteoarthritis.

The Following Samples were Evaluated
(1) PVD 06 [Boswellia serrata (300 mg)+Apium graveolens (Celery) seed extract (250 mg)]
(2) PVD 02 Group: (Boswellia serrata extract capsules (300 mg)
(3) PVD 03 Group: Apium graveolens (Celery) seed extract (250 mg)
(4) Placebo Group: Placebo capsules.
   Treatment Arms with Dosage

PVD 06 Group: One PVD 06 capsule twice a day for 90 days.

PVD 02 Group: One PVD 06/01 capsule twice a day for 90 days.

PVD 03 Group: One PVD 03 capsule twice a day for 90 days.

Placebo Group: One Placebo capsule twice a day for 90 days.

### Evaluation

1.Changes in WOMAC A, B, and C subscale scores for pain, stiffness, and physical disability at screening, day 7, day 15, day 30, day 45, day 60, and day 90.
WOMAC-A is assessed by a Likert scale on a range from 0 (none) to 4 (extreme), with higher scores indicating higher levels of pain.
WOMAC-B for levels of stiffness and
WOMAC-C for levels of physical disability

2. Changes in physician's global assessment for pain at screening, day 1, day 3, day 7, day 15, day 30,

The primary objectives of the study were to evaluate—

1.Changes in WOMAC A, B, and C subscale scores for pain, stiffness, and physical disability at screening, day 7, day 15, day 30, day 45, day 60, and day 90.
WOMAC-A is assessed by a Likert scale on a range from 0 (none) to 4 (extreme), with higher scores indicating higher levels of pain.
WOMAC-B for levels of stiffness and
WOMAC-C for levels of physical disability

2. Changes in the physician's global assessment for pain at screening, day 1, day 3, day 7, day 15, day 30, day 45, day 60, and day 90.

3. Changes in distance covered in the six-minute walk test on day 0, day 7, day 15, day 30, day 45, day 60, and day 90

4. Changes in VAS pain scale score at screening, day 1, day 3, day 7, day 15, day 30, day 45, day 60, day 90, day 105 and day 120

5. Changes in radiological examination (X-ray) only for participants as per the discretion of the investigator, day 0 and day 90

The secondary objectives of the study were to evaluate—
1.Changes in biomarkers such as PIIANP, Human Procollagen 2 C-terminal Peptide, Cartilage Oligomeric Matrix Protein, C-Telopeptide of type II collagen (CTX-II), TNF-Alpha, IL-1, IL-6, IL-7, hsCRP, and MMPS (MMP 3) at screening and day 90.
2.Changes in the requirement of analgesic as a rescue medication at screening, day 30, day 60, day 90, day 105, and day 120.
3.Changes in gastrointestinal symptoms like heartburn, gastric discomfort, and epigastric pain on the 4-point Linkert scale at screening, day 7, day 15, day 30, day 45, day 60, and day 90.
4.Changes in KOOS scoring for quality of life at day 0 and day 90.
5.Changes in FACIT-F score at day 0 and day 90.
6.Changes in symptom grading on a 4-point Linkert scale for joint swelling, tenderness, and warmth at screening, day 1, day 3, day 7, day 15, day 30, day 45, day 60, and day 90.

Assessment of WOMAC A, B, and C subscale scores, and gastrointestinal symptoms was done at screening, day 7, day 15, day 30, day 45, day 60, and day 90.

Assessment of physicians' global assessment for pain and OA-related symptoms on the Likert scale was performed on screening, day 1, day 3, day 7, day 15, day 30, day 45, day 60, and day 90.

Assessment of the six-minute walk test was performed on day 0, day 7, day 15, day 30, day 45, day 60, and day 90.

This was a randomised, double-blind, parallel-group, comparative, multicenter, placebo-controlled, clinical study to assess the efficacy & safety of Boswellia serrata and Apium graveolens (Celery) seed extract in the management of knee osteoarthritis.

In this study, participants taking analgesics were enrolled after an appropriate washout period (at least 3 days). A total of 120 participants were randomized to either one of the following groups: PVD 02 Group: Boswellia serrata extract capsules (300 mg), PVD 06 Group: Boswellia serrata (300 mg)+Apium graveolens (Celery) seed extract (250) capsule, PVD 03 Group: Apium graveolens (Celery) extract (250 mg) capsule, Placebo Group: Placebo capsule in 1:1:1:1 ratio (30 participants in each group). The treatment duration was 90 days, and the study duration of 120 days. The efficacy of the investigational products was compared between the groups.

Concomitant diseases/medication assessment was performed on screening.

Assessment of WOMAC A, B, and C subscale scores, and gastrointestinal symptoms was done at screening, day 7, day 15, day 30, day 45, day 60, and day 90.

Assessment of physicians' global assessment for pain and OA-related symptoms on the Likert scale was performed on screening, day 1, day 3, day 7, day 15, day 30, day 45, day 60, and day 90.

Assessment of the six-minute walk test was performed on day 0, day 7, day 15, day 30, day 45, day 60, and day 90.

Assessment of VAS pain score was done on screening, day 1, day 3, day 7, day 15, day 30, day 45, day 60, day 90, day 105, and day 120. Day 105 and day 120 follow-ups were performed telephonically.

Assessment of haematological & biochemical investigations and biomarkers was performed on screening and day 90. Assessment of X-ray, KOOS questionnaire score, FACIT-F score, and ECG examination was done at the baseline and day 90.

The need for analgesics as a rescue medication was assessed on screening, day 30, day 60, day 90, day 105, and day 120.

Safety of the investigational treatment in terms of adverse events (AEs), and serious adverse events (SAEs) was assessed from randomisation to day 90. Treatment compliance was assessed on day 30, day 60, and day 90.

Assessment of WOMAC A, B, and C subscale scores, and gastrointestinal symptoms was done at screening, day 7, day 15, day 30, day 45, day 60, and day 90.

The primary objectives of the study were to evaluate—

1.Changes in WOMAC A, B, and C subscale scores for pain, stiffness, and physical disability at screening, day 7, day 15, day 30, day 45, day 60, and day 90.
WOMAC-A is assessed by a Likert scale on a range from 0 (none) to 4 (extreme), with higher scores indicating higher levels of pain.
WOMAC-B for levels of stiffness and
WOMAC-C for levels of physical disability

2. Changes in the physician's global assessment for pain at screening, day 1, day 3, day 7, day 15, day 30, day 45, day 60, and day 90.

3. Changes in distance covered in the six-minute walk test on day 0, day 7, day 15, day 30, day 45, day 60, and day 90

4. Changes in VAS pain scale score at screening, day 1, day 3, day 7, day 15, day 30, day 45, day 60, day 90, day 105 and day 120

5. Changes in radiological examination (X-ray) only for participants as per the discretion of the investigator, day 0 and day 90

The need for analgesics as a rescue medication was assessed on screening, day 30, day 60, day 90, day 105, and day 120.

All analyses were done using SPSS Version 10.0. All data were summarised with descriptive statistics (number of participants, mean, standard deviation, minimum, median, and maximum) for continuous endpoints and frequency and percentage for categorical endpoints. Primary and secondary efficacy endpoints were intended to be analysed using the PP (per protocol) population and Safety variables as per mITT (modified intention-to-treat).

Primary and secondary efficacy endpoints were intended to be analyzed using the PP (per protocol) population and Safety variables as per mITT (modified intention-to-treat).

### Investigational Product Description

Active Ingredients of Boswellia serrata Extract Capsules (PVD 02)

| Sr. No. | Name of the Ingredient | Composition |
|---|---|---|
| 1. | Boswellia serrata extract | 300 mg |

Active Ingredients of Boswellia serrata Extract+Apium graveolens Seed Extract Capsule 550 mg (PVD 06)

| Sr. No. | Name of the Ingredient | Composition |
|---|---|---|
| 1. | Boswellia serrata extract | 300 mg |
| 2. | Apium graveolens (Celery) seed extract | 250 mg |

Active Ingredients of Apium graveolens Seed Extract Capsule 250 mg (PVD 03)

| Sr. No. | Name of the Ingredient | Composition |
|---|---|---|
| 1. | Apium graveolens (Celery) seed extract | 250 mg |

### Ingredients of a Placebo Capsule

| Sr. No. | Name of the Ingredient | Composition |
|---|---|---|
| 1. | Dextrin | 500 mg |

Inactive Ingredients: Hydroxypropyl methylcellulose, Dextrin

### Assessment of the WOMAC Score Between Groups

The WOMAC questionnaire comprises three subscales: pain, stiffness, and physical function. Each question or activity is assessed on a difficulty scale ranging from 0 (None), 1 (Slight), 2 (Moderate), 3 (Very), and 4 (Extremely), with higher scores indicating greater difficulty and poorer function. The total score is obtained by summing the response scores for each subscale. Improvement is reflected as a reduction in the score.

Assessment of changes in total WOMAC score between groupsAssessment of changes in the requirement of analgesics as a rescue medication

Changes in Total WOMAC score

| | Boswellia | | | |
|---|---|---|---|---|
| | serrata + Celery | Boswellia | | |
| | seed Extract | serrata | | Celery seed |
| | (High Dose) | Extract | | extract |
| | (300 + 250 = | (300 mg) | | (250 mg) |
| Groups | 550 mg) (PVD 06) | (PVD 02) | Placebo | (PVD 03) |
| Total | 23.37 ± 2.58 | 31.39 ± 2.94 | 61.43 ± 5.30 | 43.00 ± 3.79 |
| WOMAC | % Improvement | % Improvement | % Improvement | % Improvement |
| | (64.00%) | (51.03%) | (4.41%) | (35.17%) |
| P value | <0.001 | <0.001 | 0.001 | <0.001 |

Data is represented as Mean±S.D.

### Assessment of Changes in WOMAC Score for Stiffness, Pain and Physical Function Between Groups

Changes in WOMAC score

| | Boswellia | | | |
|---|---|---|---|---|
| | serrata + | | | |
| | Celery seed | Boswellia | | |
| | Extract | serrata | | Celery Seed |
| | (High Dose) | Extract | | extract |
| Groups | (PVD 06) | (PVD 02) | Placebo | (PVD 03) |
| Pain (A) | 3.17 ± 1.32 | 6.55 ± 1.15 | 13.50 ± 1.41 | 8.25 ± 1.48 |
| | % Improvement | % Improvement | % Improvement | % Improvement |
| | (78.2%) | (54.4%) | (6.9%) | (44.07%) |
| P value | <0.001 | <0.001 | <0.001 | <0.001 |
| Stiffness | 1.13 ± 0.73 | 2.32 ± 0.54 | 4.73 ± 1.08 | 3.17 ± 0.94 |
| (B) | % Improvement | % Improvement | % Improvement | % Improvement |
| | (78.1%) | (52%) | (−0.7%) | (34.48%) |
| P value | <0.001 | <0.001 | 0.856 | 0.002 |
| Physical | 19.07 ± 2.35 | 22.52 ± 2.41 | 43.20 ± 4.25 | 31.58 ± 2.47 |
| Function | % Improvement | % Improvement | % Improvement | % Improvement |
| (C) | (57.8%) | (49.9%) | (4.1%) | (32.44%) |
| P Value | <0.001 | <0.001 | 0.003 | <0.001 |

Data is represented as Mean±S.D.

### Assessment of Changes in VAS Pain Score

Changes in VAS pain score

| | Boswellia | | | |
|---|---|---|---|---|
| | serrata + | | | |
| | Celery seed | Boswellia | | |
| | Extract | serrata | | Celery seed |
| | (High Dose) | Extract | | extract |
| Groups | (PVD 06) | (PVD 02) | Placebo | (PVD 03) |
| VAS | 2.1 ± 0.87 | 3.1 ± 0.54 | 6.2 ± 0.91 | 4.17 ± 0.39 |
| Pain | % Improvement | % Improvement | % Improvement | % Improvement |
| | (67.71%) | (50.26%) | (7.50%) | (28.57%) |
| P value | <0.001 | <0.001 | 0.003 | <0.001 |

### Assessment of Serum Biomarkers Between Groups

Assessment of Biomarkers

| | | | | |
|---|---|---|---|---|
| | Boswellia | | | |
| | serrata + | | Placebo/ | |
| | Celery seed | Boswellia | Rescue | |
| | Extract | serrata | medicine | Celery Seed |
| | (Synergism) | extract | Celecoxib | Extract |
| Groups | (PVD 06) | (PVD 02) | (100 mg b.i.d) | (PVD 03) |

IL6 (pg/ml)

| | | | | |
|---|---|---|---|---|
| Screening | 9.58 ± 4.14 | 9.38 ± 4.71 | 9.84 ± 3.17 | 10.32 ± 2.81 |
| Day 90 | 3.19 ± 1.48 | 3.65 ± 1.61 | 7.85 ± 3.12 | 8.93 ± 2.16 |
| % change | 66.69 | 61.08 | 20.19 | 13.46 |

hs-CRP (ug/ml)

| | | | | |
|---|---|---|---|---|
| Screening | 5.05 ± 0.64 | 5.94 ± 1.24 | 5.48 ± 1.101 | 5.96 ± 1.80 |
| Day 90 | 2.22 ± 1.07 | 3.01 ± 1.30 | 5.58 ± 2.49 | 5.46 ± 2.19 |
| % change | 55.95 | 49.39 | 1.88 | 8.93% |

TNF-alpha (pg/ml)

| | | | | |
|---|---|---|---|---|
| Screening | 3.61 ± 0.55 | 3.77 ± 1.10 | 3.99 ± 0.74 | 4.17 ± 1.08 |
| Day 90 | 2.52 ± 1.09 | 3.20 ± 1.56 | 4.00 ± 1.19 | 3.70 ± 0.93 |
| % change | 30.30 | 15.22 | 0.34 | 11.27% |

Serum CTX-2 (ng/ml)

| | | | | |
|---|---|---|---|---|
| Screening | 10.03 ± 1.71 | 10.90 ± 1.81 | 10.28 ± 1.87 | 10.90 ± 1.81 |
| Day 90 | 5.88 ± 1.00 | 6.62 ± 0.78 | 10.67 ± 2.68 | 8.82 ± 0.90 |
| % change | 41.40 | 39.26 | 3.77 | 24.48% |

ESR (mm/hr)

| | | | | |
|---|---|---|---|---|
| Screening | 24.7 ± 19.8 | 20.0 ± 15.1 | 22.5 ± 14.8 | 34.25 ± 15.53 |
| Day 90 | 13.5 ± 4.2 | 15.7 ± 8.2 | 23.1 ± 14.5 | 32.50 ± 13.55 |
| % change | 45.55 | 21.29 | 2.97 | 5.10 |

IL-7 (pg/ml)

| | | | | |
|---|---|---|---|---|
| Screening | 7.55 ± 2.45 | 8.38 ± 3.30 | 7.85 ± 4.83 | 8.82 ± 3.54 |
| Day 90 | 3.94 ± 1.57 | 6.37 ± 2.55 | 7.79 ± 3.48 | 7.73 ± 2.62 |
| % change | 47.78 | 24.03 | 0.76 | 12.35% |

IL-1 (pg/ml)

| | | | | |
|---|---|---|---|---|
| Screening | 10.82 ± 3.92 | 10.01 ± 3.71 | 11.57 ± 3.30 | 9.58 ± 4.52 |
| Day 90 | 7.44 ± 3.39 | 8.79 ± 3.49 | 11.01 ± 2.99 | 9.94 ± 3.59 |
| % change | 31.25 | 12.24 | 4.85 | 3.72% |

COMP (μg/ml)

| | | | | |
|---|---|---|---|---|
| Screening | 19.02 ± 3.49 | 19.27 ± 3.22 | 18.37 ± 3.34 | 17.25 ± 3.37 |
| Day 90 | 11.60 ± 3.77 | 16.20 ± 2.66 | 17.71 ± 3.55 | 15.13 ± 3.49 |
| % change | 38.99 | 15.92 | 3.58 | 12.28% |

MMP-3 (ng/ml)

| | | | | |
|---|---|---|---|---|
| Screening | 38.28 ± 9.23 | 38.46 ± 6.05 | 37.36 ± 9.81 | 41.25 ± 9.75 |
| Day 90 | 20.51 ± 6.79 | 30.21 ± 5.67 | 35.80 ± 8.44 | 38.99 ± 7.38 |
| % change | 46.43 | 21.45 | 4.17 | 5.47% |

PIIANP (ng/ml)

| | | | | |
|---|---|---|---|---|
| Screening | 11.46 ± 3.45 | 12.98 ± 4.78 | 11.37 ± 2.04 | 14.40 ± 5.04 |
| Day 90 | 16.67 ± 4.05 | 14.89 ± 3.75 | 11.57 ± 2.33 | 15.14 ± 5.18 |
| % change | 45.38 | 14.74 | 1.77 | 4.88% |

PIICP (ng/ml)

| | | | | |
|---|---|---|---|---|
| Screening | 443.14 ± 74.15 | 457.45 ± 74.80 | 452.50 ± 67.75 | 463.70 ± 62.36 |
| Day 90 | 647.13 ± 73.47 | 550.57 ± 143.87 | 481.65 ± 139.54 | 586.59 ± 121.8 |
| % change | 46.03 | 20.36 | 6.44 | 20.94% |

The knee joint pain that occurred during the study period was managed by advising participants to use rescue analgesics, which were Tab. Celecoxib as per requirement and terminated as soon as the symptoms subside, as per the discretion of the investigator. The cumulative number of participants who required rescue medication.

### Cumulative Number of Participant Requirements of Analgesic as a Rescue Medication

Cumulative number of participants requiring analgesics

| | | | | | | | % participants |
|---|---|---|---|---|---|---|---|
| | Day | Day | Day | Day | Day | Day | requiring |
| Group | 7 | 15 | 30 | 45 | 60 | 90 | analgesics |
| PVD 06 | 0 | 1 | 2 | 2 | 2 | 3 | 10.00 |
| Synergism | | | | | | | |
| PVD 03 | 3 | 3 | 4 | 4 | 6 | 8 | 26.66 |
| PVD 02 | 3 | 5 | 6 | 6 | 6 | 6 | 19.35 |
| Placebo | 4 | 8 | 16 | 18 | 21 | 22 | 73.33 |

References—The following references are incorporated herein by reference in their entirety for all purposes.
1. Hsu H, Siwiec R M. Knee Osteoarthritis. [Updated 2022 Sep. 4]. In: StatPearls [Internet]. Treasure Island (F L): StatPearls Publishing; 2023 January-. Available from: https://www.ncbi.nlm.nih.gov/books/NBK507884/2.
2. Sen R, Hurley J A. Osteoarthritis. [Updated 2022 May 1]. In: StatPearls [Internet]. Treasure Island (F L): StatPearls Publishing; 2022 January-. Available from: https://www.ncbi.nlm.nih.gov/books/NBK482326/Felson D T.
3. Hafez, Ashraf. (2018). Knee Osteoarthritis: A Review of Literature.
4. Jaiswal, Abhishek1; Goswami, Kiran1; Haldar, Partha1; Salve, Harshal Ramesh1; Singh, U.2. Prevalence of knee osteoarthritis, its determinants, and impact on the quality of life in elderly persons in rural Ballabgarh, Haryana. Journal of Family Medicine and Primary Care 10 (1): p 354-360, January 2021. DOI: 10.4103/jfmpc.jfmpc_1477_20
5. Pal C P, Singh P, Chaturvedi S, Pruthi K K, Vij A. Epidemiology of knee osteoarthritis in India and related factors. Indian J Orthop. 2016 September;50 (5): 518-522. doi: 10.4103/0019-5413.189608. PMID: 27746495; PMCID: PMC5017174.
6. Mora J C, Przkora R, Cruz-Almeida Y. Knee osteoarthritis: pathophysiology and current treatment modalities. J Pain Res. 2018 Oct. 5; 11:2189-2196. doi: 10.2147/JPR.S154002.
7. https://www.uptodate.com/contents/management-of-knee-osteoarthritis. Leticia Alle Deveza, Kim Bennell. May 1, 2023.
8. Uivaraseanu B, Vesa C M, Tit D M, Abid A, Maghiar O, Maghiar T A, Hozan C, Nechifor A C, Behl T, Patrascu J M, Bungau S. Therapeutic approaches in the management of knee osteoarthritis (Review). Exp Ther Med. 2022 May;23 (5): 328. doi: 10.3892/etm.2022.11257. Epub 2022 Mar. 15. PMID: 35386619; PMCID: PMC8972824.
9. Akuri M C, Barbalho S M, Val R M, Guiguer E L. Reflections about Osteoarthritis and Curcuma longa. Pharmacogn Rev. 2017 January-Jun;11 (21): 8-12. doi: 10.4103/phrev.phrev_54_16. PMID: 28503046; PMCID: PMC5414457.
10. Aghamohammadi, D., Dolatkhah, N., Bakhtiari, F. et al. Nutraceutical supplements in management of pain and disability in osteoarthritis: a systematic review and meta-analysis of randomized clinical trials. Sci Rep 10, 20892 (2020). https://doi.org/10.1038/s41598-020-78075-x
11. Liu X, Machado G C, Eyles J P, Ravi V, Hunter D J. Dietary supplements for treating osteoarthritis: a systematic review and meta-analysis. Br J Sports Med. 2018 February;52 (3): 167-175. doi: 10.1136/bjsports-2016-097333. Epub 2017 Oct. 10. PMID: 29018060.

We claim:

1. A herbal extract composition comprising:
a) enriched Boswellia serrata extract wherein the total Boswellic acids content is not less than 40% w/w, total tirucallic acids is not less than 15% w/w and Serratol is not less than 15% w/w; and
b) enriched Apium graveolens L. extract, wherein bergapten and seselin are in the ratio of the Boswellia serrata extract to the Apium graveolens L. extract at a range of 55: 45% w/w, and wherein the said composition exhibits a synergistic effect in the treatment of knee osteoarthritis.

2. The herbal extract composition of claim 1, wherein the said composition synergistically reduces the level of inflammatory biomarkers by 28-70% in patients suffering from knee osteoarthritis, wherein the said biomarkers are selected from the group consisting of IL-6, hs-CRP, TNF-alpha, IL-7, IL-1 and ESR.

3. The herbal extract composition of claim 1, wherein, the said composition, when administered to a patient suffering from knee osteoarthritis, synergistically reduces the level of cartilage degeneration biomarkers in the range 29% to 50% wherein the said cartilage degeneration biomarkers are selected from the group consisting of CTX-II, COMP, MMP-3, PIICP and PIIANP.

4. The herbal extract composition of claim 1, wherein the said composition, when administered to a patient suffering from knee osteoarthritis, increases the gap between the knee joints of a patient.

5. The herbal extract composition of claim 1, wherein the said composition is formulated in a dosage form selected from a group consisting of tablets and capsules.

6. The herbal extract composition of claim 1, wherein the said composition is administered to a patient suffering from knee osteoarthritis at a dose of 550 mg, comprising of 300 mg of B. serrata extract and 250 mg enriched Apium graveolens L. extract.

7. A composition comprising a Boswellia serrata extract enriched so that the ratio of total Boswellic acid content is not less than 40% w/w, tirucallic acids not less than 15% w/w, and Serratol not less than 15% w/w.

8. A composition comprising an Apium graveolens L. extract enriched so that the content of Bergapten and seselin is not less than 15% w/w.

9. A process of isolating enriched extract of Boswellia serrata oleo gum resin comprising the following steps:
(a) procuring and successive grinding of the oleo gum resin to prepare coarse powder;
(b) allowing the coarse oleo gum resin to macerate or percolate in the presence of ethyl alcohol resulting in resin:alcohol::1:4 w/w at room temperature for 24 hours;
(c) extracting the resin:alcohol::1:4 w/w at ambient temperature for 0.5 to 10 hours;
(d) repeating step (c) 5-6 times to obtain an alcoholic extract containing not less than 40% Boswellic acids;
(e) using the residue obtained after Boswellic acid extraction to enrich fraction consisting of Serratol by further extracting with Hexane on a silica gel column; and
(f) distilling the solvent layer obtained in step (e) on a rotary evaporator to obtain an extract containing not less than 40% w/w total boswellic acids.

10. A process of enriching Apium graveolens L. seed extract comprising the following steps:
(a) procuring and successive grinding of Apium graveolens L. seeds to prepare coarse powder;
(b) allowing the coarse powder to macerate or percolate in the presence of the solvent at room temperature for 24 hours;
(c) extracting the seeds with alcohol at a seeds: alcohol ratio of 1:4 at an ambient temperature for 0.5 to 10 hours;
(d) repeating step (c) 5-6 times to obtain an alcoholic extract;
(e) concentrating the said extract to isolate Apium graveolens L. extract comprising 0.60% to 5% w/w Bergapten and 3.15% to 20% w/w seselin.

11. A method of treating a subject in need of cartilage regeneration or having osteoarthritis comprising the steps of providing the herbal extract composition of claim 1, and administering an effective amount to the subject, wherein the herbal extract composition is effective in regenerating cartilage and/or ameliorating the effects of osteoarthritis.

12. The method of claim 11, wherein the osteoarthritis is of the knee.
